(19) United States
(12) Patent Application Publication
(10) Pub. No.: US 2015/0209284 A1
(43) Pub. Date: Jul. 30, 2015

(54) DUAL-TARGETING DRUG CARRIER AND METHOD FOR FABRICATING THE SAME

(71) Applicant: National Chung Cheng University, Min-Hsiung (TW)

(72) Inventors: Yung-Chih KUO, Min-Hsiung (TW); Yu-Hsuan CHANG, Huwei Township (TW)

(73) Assignee: National Chung Cheng University, Min-Hsiung (TW)

(21) Appl. No.: 14/271,961

(22) Filed: May 7, 2014

(30) Foreign Application Priority Data
Jan. 24, 2014 (TW) 103102683

(51) Int. Cl. A61K 9/14; A61K 47/32; A61K 31/704; A61K 31/7048; A61K 31/17

(52) U.S. Cl. CPC A61K 9/146; A61K 31/7048; A61K 31/17; A61K 31/704; A61K 47/32

(57) ABSTRACT

The present invention discloses a dual-targeting drug carrier and a method for fabricating the same, wherein WGA- and FA-modified MPEG-PLA nanoparticles of the carrier enable the anticancer drugs encapsulated thereinside to pass through BBB and target human glioblastoma cells. The dual-target drug carrier is fabricated in an emulsion-solvent evaporation technology and verified with an in-vitro BBB model formed of HBMECs, HAs and HBVPs. The present invention can increase the permeability of the in-vitro BBB model to the dual-target drug carrier and promote the glioblastoma-inhibition effect. Therefore, the present invention would contribute to the clinical therapy of brain cancers substantially in the future.

This application claims priority for Taiwan patent application no. 103102683 filed at Jan. 24, 2014, the content of which is incorporated by reference in its entirely.

### BACKGROUND OF THE INVENTION

1. Field of the Invention

The present invention relates to a drug delivery system, particularly to a drug carrier simultaneously targeting human blood brain barrier and human glioblastoma cells and a method for fabricating the same.

2. Description of the Related Art

Glioblastoma multiforme is an adult brain cancer, which is seen frequently and deteriorates fastest. Surgery is the primary method to threat glioblastoma multiforme. The treatments available to glioblastoma multiforme also include radiotherapy and chemotherapy. However, the blood brain barrier (BBB) inhibits anti-brain cancer drugs from entering the brain and retards the anti-brain cancer drugs from effectively curing the brain cancers. Therefore, there are drug carriers grafted with some specific ligands or antibodies, which target some specific receptors of the blood brain barrier, to promote the effect of brain cancer treatments.

In order to improve the absorption of nanoparticles in the brain following nasal administration, some scholars conjugated wheat germ agglutinin (WGA) with the thiolated protein on the surface of poly(ethylene glycol)-poly(lactic acid) (PEG-PLA) nanoparticles to target the N-acetylglucosamine of human brain microvascular endothelial cells (HBMECs) and encapsulated the PEG-PLA nanoparticles with a fluorescent material coumarin for analysis. The result of analyzing the fluorescent material in the brain showed that the content of the WGA functionalized nanoparticles was 2-3 folds in brain tissues than the unmodified ones.

In addition, folate receptors are highly expressed on the surface of many human cancers, and the density thereof increases during deterioration of cancer cells. Some researchers have used folate-monomethoxy poly(ethylene glycol)-b-poly(lactide) (folate-MPEG-PLA) nanoparticles to carry an anticancer drug Paclitaxel. They compared the availability of folic acid for two types of cancer cells HeLa cells and glioma C6 cells in in-vitro cellular uptake and toxicity tests and proved that folic acid-modified PLA nanoparticles did significantly increase cancer cell intake.

The intelligent nanoparticles being developed currently are mostly single-targeting drug carriers. The Inventors had been particularly dedicated to studying multi-targeting drug carriers and finally developed a simple and effective dual-targeting drug carrier for treating hard-to-cure glioblastoma and a method for fabricating the same. The present invention can function as a BBB-permeating drug delivery system and apply to promoting the effect of brain cancer therapy.

### SUMMARY OF THE INVENTION

The primary objective is to provide a dual-targeting drug carrier and a method for fabricating the same, wherein folic acid (FA) and wheat germ agglutinin (WGA) are grafted on the surface of methoxy poly(ethylene glycol)-poly(ε-caprolactone) nanoparticle (MPEG-PLA nanoparticles) to form a dual-targeting drug carrier effectively permeating BBB and targeting human glioblastoma cells, whereby the harm of anticancer drugs to normal cells is decreased and the effect of treating glioblastoma is increased.

To achieve the abovementioned objective, the present invention proposes a dual-targeting drug carrier, which comprises a plurality of methoxy poly(ethylene glycol)-poly(ε-caprolactone) nanoparticle (MPEG-PLA nanoparticles) each encapsulating at least one anticancer drug, folic acid (FA), and wheat germ agglutinin (WGA), wherein FA and WGA are grafted on the surface of the MPEG-PLA nanoparticles, whereby the anticancer drug encapsulated inside the MPEG-PLA nanoparticles can permeate the blood brain barrier (BBB) and target human glioblastoma cells.

The present invention also proposes a method for fabricating a dual-targeting drug carrier, which comprises steps: adding MPEG-PLA nanoparticles encapsulating or not encapsulating at least one anticancer drug and a PEG-phospholipid modifier group into an organic solvent, and agitating them until they are dissolved to obtain a first solution; adding a surfactant to emulsify the first solution and obtain a second solution; adding ultrapure water to the second solution, and agitating the second solution to evaporate the organic solvent from the second solution and obtain a third solution, and centrifugally processing the third solution to obtain a precipitation; adding N-hydroxysuccinimide sodium salt (NHS) and (1-(3-dimethylaminopropyl)-3-ethylcarbodiimide hydrochloride) (EDC) to the precipitation to activate the carboxylic groups on the surface of the MPEG-PLA nanoparticles, and using a Millipore filter to remove the residual reaction medium from the precipitation; adding wheat germ agglutinin (WGA) to the precipitation to undertake a reaction and make WGA bonded to the surface of the MPEG-PLA nanoparticles to obtain a first product, and using a Millipore filter to remove the unbonded WGA from the first product; adding folic acid (FA) to the first product to undertake a reaction and make FA bonded to the surface of the MPEG-PLA nanoparticles to obtain a second product, and using a Millipore filter to remove the unbonded FA from the second product and obtain the dual-targeting drug carrier of the present invention.

The dual-targeting drug carrier and the method for fabricating the same of the present invention are characterized in that WGA and FA grafted on the surface of the MPEG-PLA nanoparticles respectively target the N-acetylglucosamine of the in-vitro BBB model and the folic receptors of human glioblastoma cells. Thus, the dual-targeting drug carrier can permeate the in-vitro BBB model and target the folic receptors of human glioblastoma cells. Therefore, the present invention can increase the BBB permeability to the drug carrier and target human glioblastoma cells. The dual-targeting drug carrier of the present invention is easy to fabricate and able to target human glioblastoma cells highly specifically.

Below, the embodiments are described in detail to make easily understood the objectives, technical contents, characteristics and accomplishments of the present invention.

### BRIEF DESCRIPTION OF THE DRAWINGS

FIG. 1 shows a flowchart of a method for fabricating a dual-targeting drug carrier according to one embodiment of the present invention;

FIG. 2 shows XPS spectra of different formulations of particles;

FIGS. 3(a)-3(d) show TEM micrographs of different formulations of particles;

FIG. 4A shows the permeabilities of the in-vitro BBB model to different formulations of particles containing ETO;

FIG. 4B shows the glioblastoma-inhibition effects of different formulations of particles containing ETO after they have permeated the in-vitro BBB model;

FIG. 5A shows the permeabilities of the in-vitro BBB model to different formulations of particles containing BCNU injection;

FIG. 5B shows the glioblastoma-inhibition effects of different formulations of particles containing BCNU injection after they have permeated the in-vitro BBB model;

FIG. 6A shows the permeabilities of the in-vitro BBB model to different formulations of particles containing DOX injection;

FIG. 6B shows the glioblastoma-inhibition effects of different formulations of particles containing DOX injection after they have permeated the in-vitro BBB model;

FIGS. 7(a)-7(e) show immunofluorescent staining images taken by a confocal microscope to observe the intakes of different formulations of particles by HBMECs and human glioblastoma cells; and

FIGS. 8(a)-8(e) show immunofluorescent staining images taken by a confocal microscope to observe the targeting effects of different formulations of particles to the human glioblastoma cells.

### DETAILED DESCRIPTION OF THE INVENTION

The present invention proposes a dual-targeting drug carrier, which comprises a plurality of MPEG-PLA nanoparticles. Each MPEG-PLA nanoparticle encapsulates at least one anticancer drug, such as at least one of etoposide, carmustine and doxorubicin. The surfaces of the MPEG-PLA nanoparticles are grafted with wheat germ agglutinin (WGA) and folic acid (FA). Thereby, the anticancer drugs inside the MPEG-PLA nanoparticles can permeate BBB and target human glioblastoma cells (U87MG cells).

The method for fabricating the dual-targeting drug carrier of the present invention and the verification thereof will be described thereinafter. In one embodiment, an emulsion-solvent evaporation technology is used to fabricate the dual-targeting drug carrier of the present invention. Human brain microvascular endothelial cells (HBMECs), human astrocytes (HAs) and human brain vascular pericytes (HBVPs) are used to construct an in-vitro BBB model simulating the transmission mechanism of drugs in the brain, whereby to evaluate the BBB permeating capabilities and the glioblastoma cell-targeting effects of different formulations of particles encapsulating anticancer drugs thereinside. Further, immunofluorescence is used to observe HBMECs and human glioblastoma cells absorbing the particles.

### A. Processes and Conditions

### I. Fabrication of Dual-Targeting Drug Carrier:

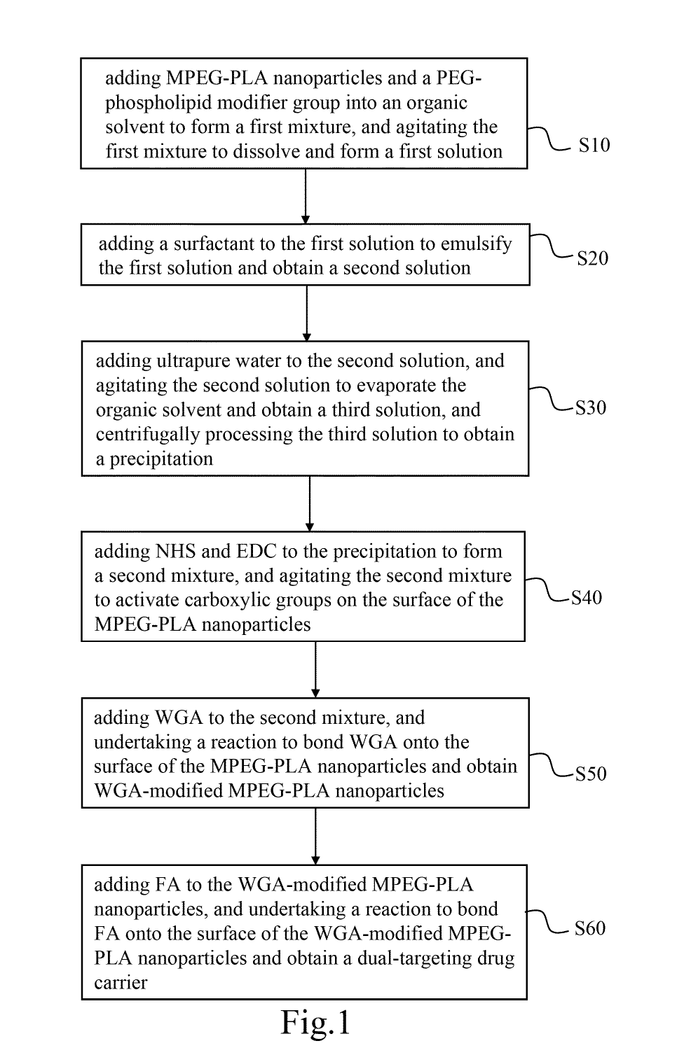

Refer to FIG. 1 for a flowchart of a method for fabricating a dual-targeting drug carrier according to one embodiment of the present invention. The steps of the method are described in detail below.
(1) In Step S10, add 10 mg of MPEG-PLA nanoparticles and a PEG-phospholipid modifier group, which are mixed by a molar ratio of 1:0.25, to 0.5 mg of an organic solvent, to form a first mixture, and agitate the first mixture until the first mixture are dissolved to form a first solution. The MPEG-PLA nanoparticles encapsulate or do not encapsulate one or more anticancer drugs. In one embodiment, the PEG-phospholipid modifier group is DSPE-PEG(2000)-CA, and the organic solvent is chloroform.
(2) In Step S20, gradually add 1 ml of a surfactant-containing aqueous solution to the first solution, and agitate the first solution at a rotation speed of 1200 rpm for 5 minutes to emulsify the first solution and obtain a second solution. In one embodiment, the surfactant is Pluronic F127.
(3) In Step S30, add 9 ml of ultrapure water to the second solution, and agitate the second solution at a rotation speed of 1200 rpm for 3 hours to evaporate the organic solvent from the second solution and obtain a third solution, and centrifugally process the third solution by 2465×g for 20 minutes to obtain a precipitation from the bottom.
(4) In Step S40, add 100 μl of a 0.05%(w/v) N-hydroxysuccinimide sodium salt (NHS) solution and 100 μl of 0.1% (w/v) (1-(3-dimethylaminopropyl)-3-ethylcarbodiimide hydrochloride) (EDC) solution to the precipitation to form a second mixture, and agitate the second mixture at a rotation speed of 200 rpm and a temperature of 4° C. for 3 hours to activate the carboxylic groups on the surface of the MPEG-PLA nanoparticles, and use a Millipore filter to remove the residual reaction medium from the second mixture.
(5) In Step S50, add 100 μl of 0.1% (w/v) WGA solution to the second mixture, and undertake a reaction at a temperature of 4° C. for 5 hours to make WGA bonded to the surface of the MPEG-PLA nanoparticles and obtain a first product, and use a Millipore filter to remove the unbonded WGA from the first product and obtain WGA-modified MPEG-PLA nanoparticles.
(6) In Step S60, add 50 μl of a 0.05% (w/v) FA to the WGA-modified MPEG-PLA nanoparticles, and undertake a reaction at a temperature of 4° C. for 5 hours to make FA bonded to the surface of the MPEG-PLA nanoparticles and obtain a second product, and use a Millipore filter to remove the unbonded FA from the second product and obtain the dual-targeting drug carrier of the present invention.

In the abovementioned embodiment, 82.5% of WGA and 84.6% of FA are grafted on the surface of the MPEG-PLA nanoparticles; the MPEG-PLA nanoparticles of the obtained dual-targeting drug carrier have a diameter of about 150 nm; the weight ratio of WGA and FA bonded to the surface of the MPEG-PLA nanoparticles is 4:1.

### II. Construction of In-Vitro BBB Model:

(1) Flip the bottom side of the PET membrane of the transwell having been processed beforehand, and inject the suspension liquids of HBVPs and HAs into the transwell by a ratio of 1:2, and let the cells undertake attachment for 1 hour. Next, inject the suspension liquid of HBMECs into the top side of the PET membrane of the transwell. Maintain the culture mediums of the transwell and the 24-well plate at the same level, and place the transwell in a CO₂ incubator having a temperature of 37° C. and a relative humidity of 95%. The seeding densities are all 4×10⁵ cells/cm². As long as HBMECs, HBVPs and HAs are grown to 80% of the desired density, the culture medium of the lower chamber of the 24-well plate of the transwell is changed to be a medium containing ACM2 and PCM2 by a ratio of 1:1. Next, place the transwell in a CO₂ incubator having a temperature of 37° C. and a relative humidity of 95%. Then, change the culture medium every day, and observe the state of cell growth. After HBMECs, HBVPs and HAs have been cultured for 7 days, they are used in experiments.
(2) Place a suspension liquid of the dual-targeting drug carrier, which contains an anticancer drug having a concentration of 0.25 mg/ml, into the transwell of the co-culture system of HBMECs/HAs/HBVPs. Maintain the culture mediums of the transwell and the 24-well plate at the same level, and place the transwell in a CO₂ incubator having a temperature of 37° C. and a relative humidity of 95%. Then, take samples from the 24-well plate at the preset time points.

### III. Experiment Preparation for Dual-Targeting Drug Carrier:

(1) Culture U87MG cells in a 96-wll microtiter plate at a seeding density of 5×10³ cells/well. Inject 100 μl of a culture medium into each well, and place the microtiter plate in a CO₂ incubator having a temperature of 37° C. and a relative humidity of 95% for 24 hours. Then, remove the culture medium.
(2) After 3 hours of penetration, add the dual-targeting drug carrier containing an anticancer drug into each well, and place the microtiter plate in a CO₂ incubator having a temperature of 37° C. and a relative humidity of 95% for 24 hours.

### B. Experiments

### I. Dual-Targeting Drug Carrier:

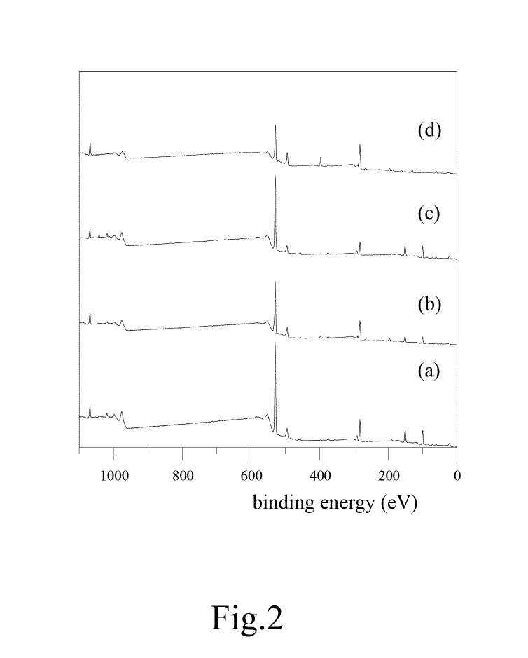

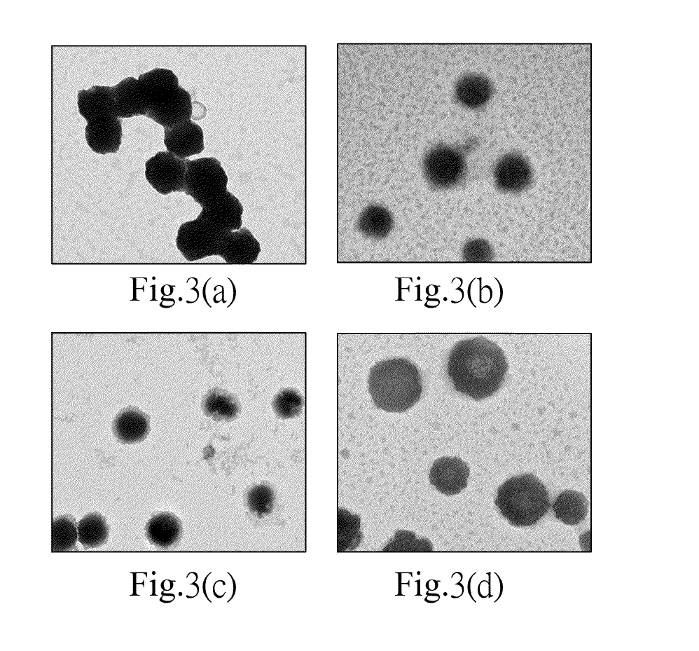

Use X-ray photoelectron spectroscopy (XPS) to analyze the elements on the surfaces of different formulations of particles, and use the content of nitrogen element to verify whether WGA and FA are bonded to the surface of MPEG-PLA nanoparticles. Use a transmission electron microscope (TEM) to observe the surfaces of different formulations of particles. The examination results are shown in FIG. 2 and FIG. 3 and described thereinafter.

### II. Permeability of In-Vitro BBB Model:

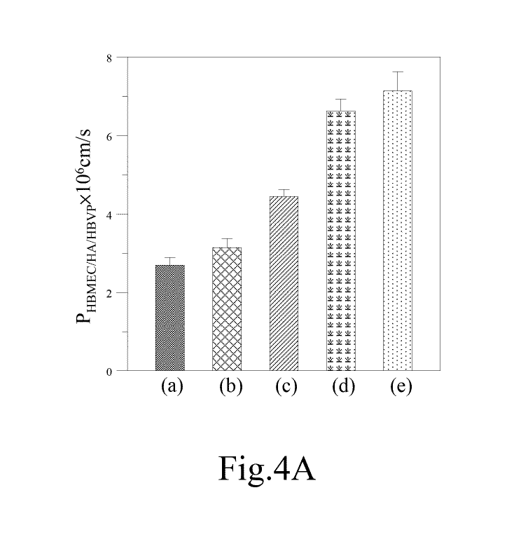

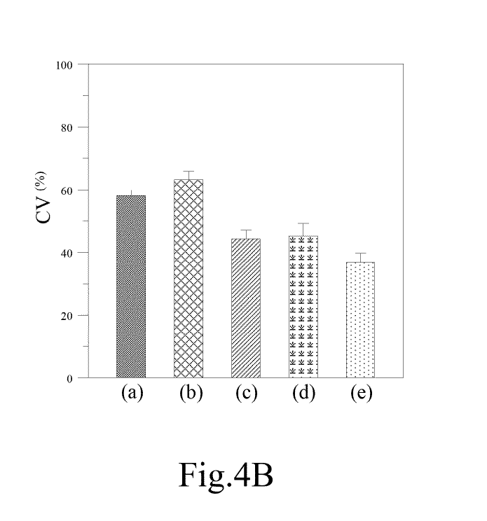

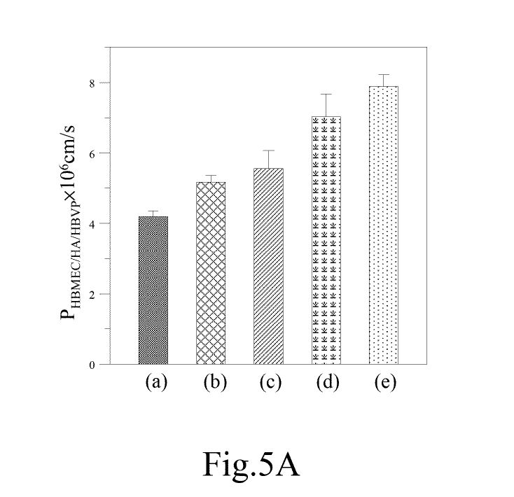

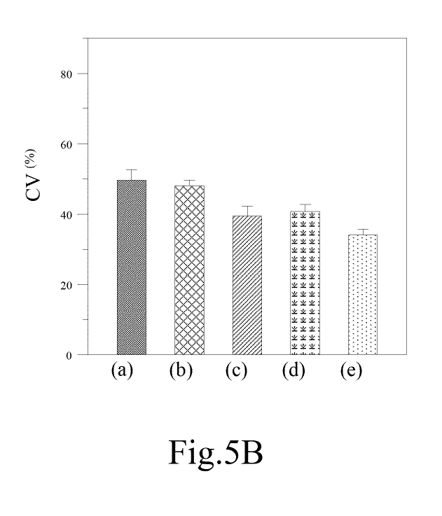

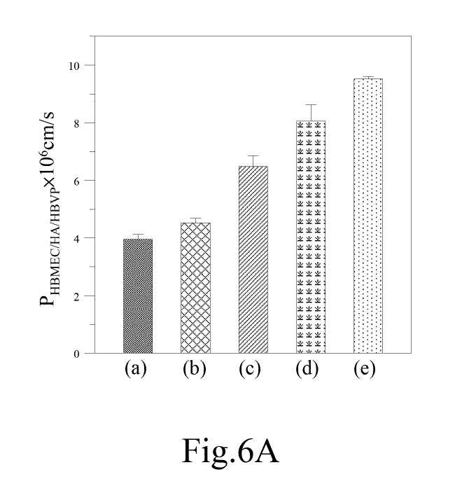

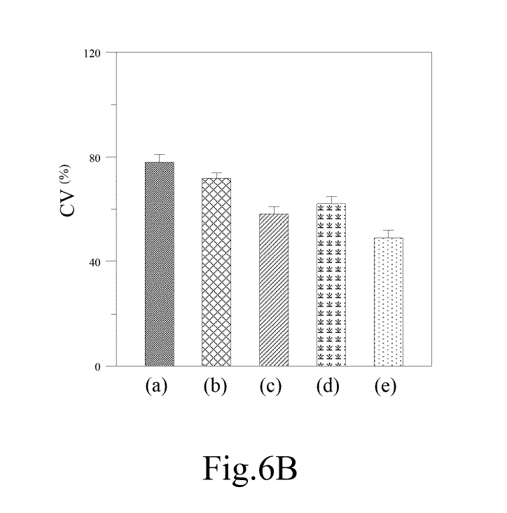

Use ELISA (Enzyme-Linked ImmunoSorbent Assay) to respectively analyze the concentrations of ETO (etoposide) injection, BCNU (carmustine) and DOX (doxorubicin) injection at the wavelengths of 284 nm, 230 nm and 485 nm. The examination results are shown in FIG. 4A, FIG. 5A and FIG. 6A and described thereinafter.

The equation of the permeability coefficient can be expressed by

$$\frac{1}{{P}_{coc}} = \frac{1}{{P}_{e}} - \frac{1}{{P}_{m}}$$

$${P}_{i} = \frac{J \text{/} A}{Δ C} = {V}_{x} ( \frac{{dC}_{x} \text{/} dt}{A Δ C} )$$

wherein
i=e or m
P_{coc}: permeability of a material passing through HBMECs/HAs
Pₑ: permeability of a material passing through HBMECs/HAs grafted on a PET membrane
Pₘ: permeability of a material passing through a PET membrane
J/A: mass flux of a material from a donor chamber to a receiver chamber
ΔC: concentration difference between the donor chamber and the receiver chamber
Vᵣ: volume of the receiver chamber
Cᵣ: concentration of a material in the receiver chamber
t: time
A: area for material permeation

### III. Glioblastoma-Inhibition Effect of Dual-Targeting Drug Carrier:

(1) Culture U87MG cells in a 96-well microtiter plate at a seeding density of 5×10³ cells/well. Inject 100 μl of a culture medium into each well, and place the microtiter plate in a CO₂ incubator having a temperature of 37° C. and a relative humidity of 95% for 24 hours. Then, remove the culture medium.
(2) After 3 hours of penetration, add the samples obtained from the abovementioned step into the wells, and place the microtiter plate in a CO₂ incubator having a temperature of 37° C. and a relative humidity of 95% for 24 hours.
(3) Remove the culture medium containing MPEG-PLA nanoparticles or anticancer drugs. Then, add a mixture of 150 μl of a new culture medium and 50 μl of XTT to each well, and place the microtiter plate in a CO₂ incubator having a temperature of 37° C. and a relative humidity of 95% for 4 hours.
(4) If the test agent presents an orange color after reaction, use ELISA to obtain the absorption value at a wavelength of 450 nm and calculate the cell viability CV (%).

The examination results of the glioblastoma-inhibition effect of the dual-targeting drug carrier are shown in FIG. 4B, FIG. 5B and FIG. 6B and described thereinafter.

### IV. Particle Intakes by HBMECs and U87MG Cells (Using a Confocal Microscope)

(1) Place cover slips in the 24-well plate; culture HBMECs at a seeding density of 4×10⁴ cells/cm² and U87MG cells at a seeding density of 2×10⁴ cells/cm² in wells containing 0.5 ml of a culture medium; and place the 24-well plate in a CO₂ incubator having a temperature of 37° C. and a relative humidity of 95% for 24 hours.
(2) After cellular attachment is done, use a phosphoric acid buffer solution (DPBS) to flush the cells 3 times; add 0.5 ml of a culture medium containing nanoparticles having fluorescein isothiocyanate (FITC) to the wells, and culture the cells for 3 hours; flush the cells with DPBS 3 times; add a 4% (v/v) formalin solution to the wells, and let reaction persist for 10 minutes at an ambient temperature; flush the cells with DPBS 3 times; add a DPBS solution containing 0.5% (v/v) nonionic surfactant Triton X-100 to the wells, and keep the 24-well plate still for 10 minutes; flush the cells with DPBS 3 times; add 200 μl of a serum block solution to the wells, and let reaction persist for 10 minutes at an ambient temperature.
(3) Add to the wells an anti-O-linked N-acetylglucosamine antibody and an anti-folate binding protein antibody, which have been diluted by an antibody dilute solution 100 times and 150 times respectively. Undertake reaction overnight at a temperature of 4° C. Then, flush the cells with DPBS 3 times.
(4) Remove the old culture medium; add to the wells an anti-mouse IgG (H&L), which has been diluted by an antibody dilute solution 200 times; protect the wells from light, and let reaction persist for 1 hour; flush the cells with DPBS 3 times; add to the well DAPI, which has been diluted 200 times, and keep the 24-well plate still for 3 minutes; flush the cells with DPBS 3 times. Then, observe the cells with a confocal laser microscope, wherein the beams for exciting red light (Rothdamine), blue light (DAPI) and green light (FITC) respectively have wavelengths of 555 nm, 350 nm and 490 nm, and wherein the excited light beams respectively have wavelengths of 565 nm, 475 nm and 520 nm.

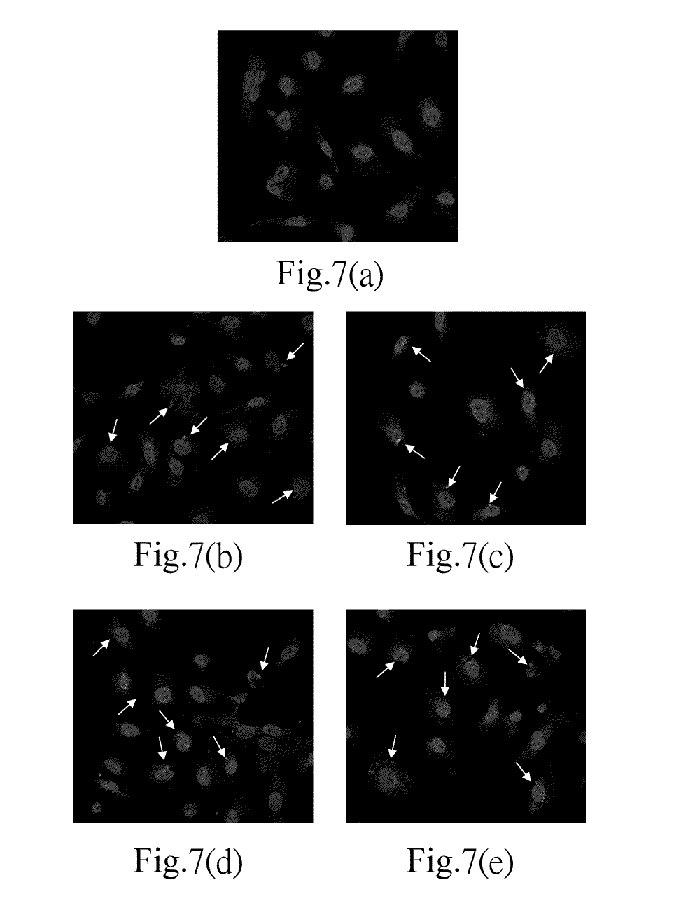

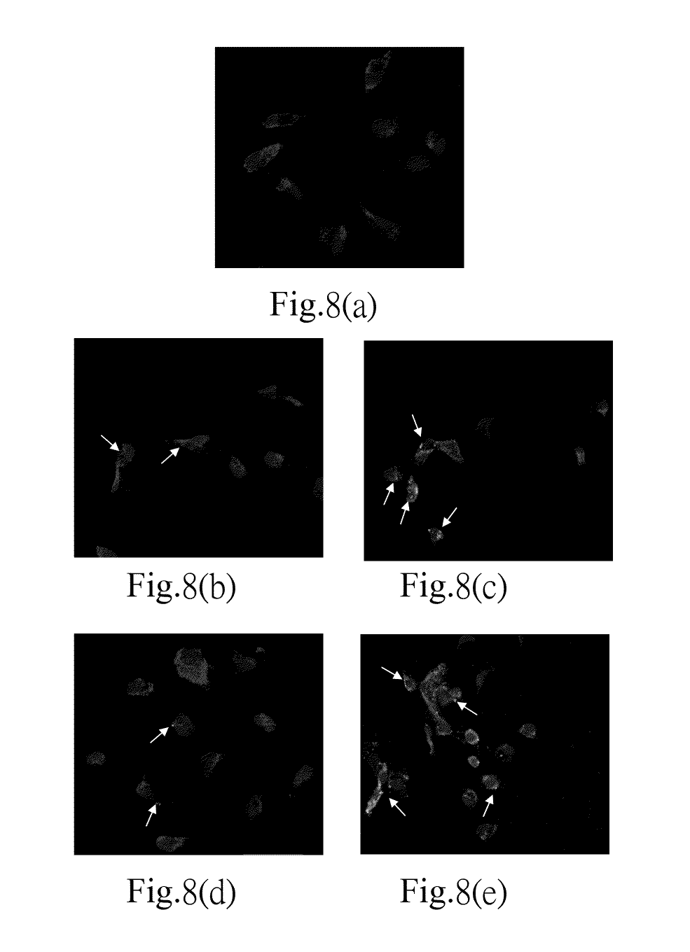

The observation results of the confocal microscope are shown in FIG. 7 and FIG. 8 and described thereinafter.

### C. Experimental Results

Refer to FIG. 2 for XPS spectra of different formulations of particles, wherein Curve (a) is an XPS spectrum of unmodified MPEG-PLA nanoparticles; Curve (b) is an XPS spectrum of FA-modified MPEG-PLA nanoparticles; Curve (c) is an XPS spectrum of WGA-modified MPEG-PLA nanoparticles; and Curve (d) is an XPS spectrum of the dual-targeting drug carrier of the present invention, i.e. the WGA- and FA-modified MPEG-PLA nanoparticles. Each of WGA and FA contains many nitrogen atoms. Therefore, the content of nitrogen atoms can be used to verify whether molecules of WGA or FA are bonded to the surface of the modified MPEG-PLA nanoparticle. The results show that the signal of nitrogen atoms does increase in the WGA- and FA-modified MPEG-PLA nanoparticles and prove that molecules of WGA or FA are indeed bonded to the surface of the modified MPEG-PLA nanoparticle.

Refer to FIGS. 3(a)-3(d) for TEM micrographs of different formulations of particles to observe whether the surfaces of particles are successfully modified, wherein FIG. 3(a) is a TEM micrograph of unmodified MPEG-PLA nanoparticles; FIG. 3(b) is a TEM micrograph of FA-modified MPEG-PLA nanoparticles; FIG. 3(c) is a TEM micrograph of WGA-modified MPEG-PLA nanoparticles; and FIG. 3(d) is a TEM micrograph of the dual-targeting drug carrier of the present invention, i.e. the WGA- and FA-modified MPEG-PLA nanoparticles. FIG. 3(a) shows that the particles are fully-black dots and indicates that the MPEG-PLA nanoparticles are unmodified. FIG. 3(d) shows that gray layers wrap the surfaces of the particles and indicates that molecules of WGA and FA are grafted on the surfaces of the MPEG-PLA nanoparticles.

Refer to FIG. 4A for the permeabilities of the in-vitro BBB model to different formulations of particles containing ETO, wherein Bar (a) is the permeability to ETO not encapsulated in particles; Bar (b) is the permeability to unmodified MPEG-PLA nanoparticles containing ETO; Bar (c) the permeability to FA-modified MPEG-PLA nanoparticles containing ETO; Bar (d) is the permeability to WGA-modified MPEG-PLA nanoparticles containing ETO; and Bar (e) is the permeability to the dual-targeting drug carrier containing ETO of the present invention, i.e. the WGA- and FA-modified MPEG-PLA nanoparticles containing ETO. The results show that the in-vitro BBB model has a higher permeability to the WGA- and FA-modified MPEG-PLA nanoparticles containing ETO.

Refer to FIG. 4B for the glioblastoma-inhibition effects of different formulations of particles containing ETO after they have permeated the in-vitro BBB model, wherein Bar (a) is the glioblastoma-inhibition effect of ETO not encapsulated in particles; Bar (b) is the glioblastoma-inhibition effect of unmodified MPEG-PLA nanoparticles containing ETO; Bar (c) the glioblastoma-inhibition effect of FA-modified MPEG-PLA nanoparticles containing ETO; Bar (d) is the glioblastoma-inhibition effect of WGA-modified MPEG-PLA nanoparticles containing ETO; and Bar (e) is the glioblastoma-inhibition effect of the dual-targeting drug carrier containing ETO of the present invention, i.e. the WGA- and FA-modified MPEG-PLA nanoparticles containing ETO. The results show that the WGA- and FA-modified MPEG-PLA nanoparticles containing ETO (i.e. the dual-targeting drug carrier containing ETO of the present invention) has the highest glioblastoma-inhibition effect.

Refer to FIG. 5A for the permeabilities of the in-vitro BBB model to different formulations of particles containing BCNU injection, wherein Bar (a) is the permeability to BCNU injection not encapsulated in particles; Bar (b) is the permeability to unmodified MPEG-PLA nanoparticles containing BCNU injection; Bar (c) the permeability to FA-modified MPEG-PLA nanoparticles containing BCNU injection; Bar (d) is the permeability to WGA-modified MPEG-PLA nanoparticles containing BCNU injection; and Bar (e) is the permeability to the dual-targeting drug carrier containing BCNU injection of the present invention, i.e. the WGA- and FA-modified MPEG-PLA nanoparticles containing BCNU injection. The results show that the in-vitro BBB model has a higher permeability to the WGA- and FA-modified MPEG-PLA nanoparticles containing BCNU injection.

Refer to FIG. 5B for the glioblastoma-inhibition effects of different formulations of particles containing BCNU injection after they have permeated the in-vitro BBB model, wherein Bar (a) is the glioblastoma-inhibition effect of BCNU injection not encapsulated in particles; Bar (b) is the glioblastoma-inhibition effect of unmodified MPEG-PLA nanoparticles containing BCNU injection; Bar (c) the glioblastoma-inhibition effect of FA-modified MPEG-PLA nanoparticles containing BCNU injection; Bar (d) is the glioblastoma-inhibition effect of WGA-modified MPEG-PLA nanoparticles containing BCNU injection; and Bar (e) is the glioblastoma-inhibition effect of the dual-targeting drug carrier containing BCNU injection of the present invention, i.e. the WGA- and FA-modified MPEG-PLA nanoparticles containing BCNU injection. The results show that the WGA- and FA-modified MPEG-PLA nanoparticles containing BCNU injection (i.e. the dual-targeting drug carrier containing BCNU injection of the present invention) has the highest glioblastoma (U87MG)-inhibition effect.

Refer to FIG. 6A for the permeabilities of the in-vitro BBB model to different formulations of particles containing DOX injection, wherein Bar (a) is the permeability to DOX injection not encapsulated in particles; Bar (b) is the permeability to unmodified MPEG-PLA nanoparticles containing DOX injection; Bar (c) the permeability to FA-modified MPEG-PLA nanoparticles containing DOX injection; Bar (d) is the permeability to WGA-modified MPEG-PLA nanoparticles containing DOX injection; and Bar (e) is the permeability to the dual-targeting drug carrier containing DOX injection of the present invention, i.e. the WGA- and FA-modified MPEG-PLA nanoparticles containing DOX injection. The results show that the in-vitro BBB model has a higher permeability to the WGA- and FA-modified MPEG-PLA nanoparticles containing DOX injection.

Refer to FIG. 6B for the glioblastoma-inhibition effects of different formulations of particles containing DOX injection after they have permeated the in-vitro BBB model, wherein Bar (a) is the glioblastoma-inhibition effect of DOX injection not encapsulated in particles; Bar (b) is the glioblastoma-inhibition effect of unmodified MPEG-PLA nanoparticles containing DOX injection; Bar (c) the glioblastoma-inhibition effect of FA-modified MPEG-PLA nanoparticles containing DOX injection; Bar (d) is the glioblastoma-inhibition effect of WGA-modified MPEG-PLA nanoparticles containing DOX injection; and Bar (e) is the glioblastoma-inhibition effect of the dual-targeting drug carrier containing DOX injection of the present invention, i.e. the WGA- and FA-modified MPEG-PLA nanoparticles containing DOX injection. The results show that the WGA- and FA-modified MPEG-PLA nanoparticles containing DOX injection (i.e. the dual-targeting drug carrier containing DOX injection of the present invention) has the highest glioblastoma-inhibition effect.

Refer to FIGS. 7(a)-7(e). In this experiment, an immunofluorescent staining technology is used to prove that HBMECs contain N-acetylglucosamine; FITC-encapsulated nanoparticles and HBMECs are co-cultured for 3 hours; then, the intake of particles by cells is observed. Please also refer to Appendix I, which shows the colored version of FIGS. 7(a)-7(e). FIG. 7(a) (parallel to Appendix I (a)) is an immunofluorescent staining image of a sample where no particle is added, wherein the black areas (corresponding to the blue areas in Appendix I) are nuclei and used to indicate the positions of cells. The gray areas (corresponding to the red areas in Appendix I) outside the black areas indicate that HBMECs contain N-acetylglucosamine and have WGA receptors. FIG. 7(b) (parallel to Appendix I (b)) is an immunofluorescent staining image of a sample where unmodified MPEG-PLA nanoparticles are added. FIG. 7(c) (parallel to Appendix I (c)) is an immunofluorescent staining image of a sample where FA-modified MPEG-PLA nanoparticles are added. FIG. 7(d) (parallel to Appendix I (d)) is an immunofluorescent staining image of a sample where WGA-modified MPEG-PLA nanoparticles are added. FIG. 7(e) (parallel to Appendix I (e)) is an immunofluorescent staining image of a sample where the dual-targeting drug carrier of the present invention, i.e. the WGA- and FA-modified MPEG-PLA nanoparticles, is added. In FIGS. 7(a)-7(e), white arrows point out the added particles (corresponding to the green areas in Appendix I). The results show that less unmodified particles are absorbed by HBMECs. As the MPEG-PLA nanoparticles in FIG. 7(d) and FIG. 7(e) has WGA on the surface thereof, the particles absorbed by HBMECs are obviously increased. The results of this experiment match the results of the permeability experiment.

Refer to FIGS. 8(a)-8(e) for immunofluorescent staining images of U87MG cells. Please also refer to Appendix II, which shows the colored version of FIGS. 8(a)-8(e). FIG. 8(a) (parallel to Appendix II (a)) is an immunofluorescent staining image of a sample where no particle is added, wherein the black areas (corresponding to the blue areas in Appendix II) are nuclei and used to indicate the positions of cells. The gray areas (corresponding to the red areas in Appendix II) outside the black areas indicate that U87MG cells contain many FA receptors. FIG. 8(b) (parallel to Appendix II (b)) is an immunofluorescent staining image of a sample where unmodified MPEG-PLA nanoparticles are added. FIG. 8(c) (parallel to Appendix II (c)) is an immunofluorescent staining image of a sample where FA-modified MPEG-PLA nanoparticles are added. FIG. 8(d) (parallel to Appendix II (d)) is an immunofluorescent staining image of a sample where WGA-modified MPEG-PLA nanoparticles are added. FIG. 8(e) (parallel to Appendix II (e)) is an immunofluorescent staining image of a sample where the dual-targeting drug carrier of the present invention, i.e. the WGA- and FA-modified MPEG-PLA nanoparticles, is added. In FIGS. 8(a)-8(e), white arrows point out the added particles (corresponding to the green areas in Appendix II). The results show that U87MG cells absorb more FA-modified MPEG-PLA nanoparticles and that FA can indeed target the FA receptors on the surface of U87MG cells.

The experimental results show that the method for fabricating a dual-targeting drug carrier can obtain an optimized particle size (about 150 nm) while the surfactant Pluronic F127 has a concentration of 1%. The TEER (transepithelial electrical resistance) measured in the in-vitro BBB model is 310±11.7 Ω×cm². Among various formulations of particles, the in-vitro BBB model has the highest permeability to the dual-targeting drug carrier, about 1.5-2.2 times the permeability to the unmodified particles. The glioblastoma-inhibition experiment shows that the anticancer drug-containing dual-targeting drug carrier of the present invention can decrease the viability of human glioblastoma cells to 37-49% within 24 hours and proves that the anticancer drug-containing dual-targeting drug carrier can effectively enhance the glioblastoma-inhibition effect after they pass through BBB. The immunofluorescent staining experiment shows that the dual-targeting drug carrier can increase the quantity of the particles absorbed by the HBMECs and human glioblastoma cells.

In conclusion, the present invention proposes a dual-targeting drug carrier and a method for fabricating the same, wherein the permeability of an in-vitro BBB model to the WGA- and FA-modified MPEG-PLA nanoparticles of the present invention is increased, and wherein the WGA- and FA-modified MPEG-PLA nanoparticles of the present invention can target human glioblastoma cells, whereby more anticancer drug can pass through the in-vitro BBB model, and whereby the glioblastoma-inhibition effect is enhanced, and whereby normal cells are less likely to be hurt. In brief, the dual-targeting drug carrier of the present invention is very suitable to transport anticancer drugs to the brain tissue and would contribute to the clinical therapy of brain cancers substantially in the future.

The embodiments described above are only to exemplify the present invention but not to limit the scope of the present invention. Any equivalent modification or variation according to the characteristic or spirit of the present invention is to be also included within the scope of the present invention.

What is claimed is:

1. A dual-targeting drug carrier comprising
a plurality of methoxy poly(ethylene glycol)-poly(ε-caprolactone) nanoparticle (MPEG-PLA nanoparticles) encapsulating at least one anticancer drug; and
wheat germ agglutinin (WGA) and folic acid (FA) modifying surface of said MPEG-PLA nanoparticles to enable said anticancer drug encapsulated by said MPEG-PLA nanoparticles to permeate a blood brain barrier (BBB) and target human glioblastoma cells.

2. The dual-targeting drug carrier according to claim 1, wherein said MPEG-PLA nanoparticles has a diameter of 150 nm.

3. The dual-targeting drug carrier according to claim 1, wherein said anticancer drug is at least one selected from a group consisting of etoposide, carmustine injection, and doxorubicin injection.

4. The dual-targeting drug carrier according to claim 1, wherein a weight ratio of said WGA and said FA is 4:1.

5. A method for fabricating a dual-targeting drug carrier, comprising steps:
adding a plurality of methoxy poly(ethylene glycol)-poly(ε-caprolactone) nanoparticle (MPEG-PLA nanoparticles) encapsulating or not encapsulating at least one anticancer drug and a poly(ethylene glycol)-phospholipid (PEG-phospholipid) modifier group into an organic solvent to form a first mixture, and agitating said first mixture until said first mixture are dissolved to form a first solution;
adding a surfactant to said first solution to emulsify said first solution and obtain a second solution;
adding ultrapure water to said second solution, and agitating said second solution to evaporate said organic solvent from said second solution and obtain a third solution, and centrifugally processing said third solution to obtain a precipitation;
adding N-hydroxysuccinimide sodium salt (NHS) and (1-(3-dimethylaminopropyl)-3-ethylcarbodiimide hydrochloride) (EDC) to said precipitation to form a second mixture, and agitating said second mixture to activate carboxylic groups on surface of said MPEG-PLA nanoparticles, and using a Millipore filter to remove residual reaction medium from said second mixture;
adding wheat germ agglutinin (WGA) to said second mixture, and undertaking a reaction to make said WGA bonded to surface of said MPEG-PLA nanoparticles and obtain a first product, and using a Millipore filter to remove unbonded said WGA from said first product and obtain WGA-modified MPEG-PLA nanoparticles; and
adding folic acid (FA) to said WGA-modified MPEG-PLA nanoparticles, and undertaking a reaction to make FA bonded to surface of said WGA-modified MPEG-PLA nanoparticles and obtain a second product, and using a Millipore filter to remove unbonded said FA from said second product and obtain a dual-targeting drug carrier.

6. The method for fabricating a dual-targeting drug carrier according to claim 5, wherein a molar ratio of said MPEG-PLA nanoparticles and said PEG-phospholipid) modifier group is 1:0.25.

7. The method for fabricating a dual-targeting drug carrier according to claim 5, wherein said PEG-phospholipid modifier group is DSPE-PEG(2000)-CA, and said organic solvent is chloroform, and said surfactant is Pluronic F127.

8. The method for fabricating a dual-targeting drug carrier according to claim 5, wherein in said step of adding NHS and EDC to said precipitation, said second mixture is agitated at a temperature of 4° C. for 3 hours.

9. The method for fabricating a dual-targeting drug carrier according to claim 5, wherein in said step of adding WGA to said second mixture, said reaction is undertaken at a temperature of 4° C. for 5 hours.

10. The method for fabricating a dual-targeting drug carrier according to claim 5, wherein in said step of adding FA to said WGA-modified MPEG-PLA nanoparticles, said reaction is undertaken at a temperature of 4° C. for 5 hours.

11. The method for fabricating a dual-targeting drug carrier according to claim 5, wherein said MPEG-PLA nanoparticles has a diameter of 150 nm.

12. The method for fabricating a dual-targeting drug carrier according to claim 5, wherein said anticancer drug is at least one selected from a group consisting of etoposide, carmustine injection, and doxorubicin injection.

13. The method for fabricating a dual-targeting drug carrier according to claim 5, wherein a weight ratio of said WGA and said FA is 4:1.
